(19) United States
(12) Patent Application Publication
(10) Pub. No.: US 2021/0228198 A1
(43) Pub. Date: Jul. 29, 2021

(54) SURGICAL ACCESS DEVICE WITH ADJUSTABLE LENGTH

(71) Applicant: Covidien LP, Mansfield, MA (US)

(72) Inventors: Nicolette R. LaPierre, Windsor Locks, CT (US); Jacob C. Baril, Norwalk, CT (US); Matthew A. Dinino, Newington, CT (US)

(21) Appl. No.: 16/774,241

(22) Filed: Jan. 28, 2020

(51) Int. Cl. A61B 17/02

(52) U.S. Cl. CPC A61B 17/0218; A61B 2017/00128

(57) ABSTRACT

A surgical access device includes a cannula body and a sleeve. The cannula body includes a housing, an elongated portion extending distally from the housing, and at least one pin extending radially outward from the elongated portion. The sleeve is disposed in mechanical cooperation with the elongated portion of the cannula body, and defines a track for slidingly engaging the at least one pin of the cannula body. The sleeve is slidable along the longitudinal axis of the elongated portion relative to the cannula body between a first position corresponding to a first length of the surgical access device and a second position corresponding to a second length of the surgical access device.

### BACKGROUND

### Technical Field

The present disclosure relates to a surgical access device. More particularly, the present disclosure relates to a surgical access device having an adjustable length.

### Background of Related Art

Endoscopic and laparoscopic minimally invasive procedures have been used for introducing medical devices inside a patient and for viewing portions of the patient's anatomy. To view a desired anatomical site, a surgeon may insert a rigid or flexible endoscope inside the patient to render images of the anatomical site.

Typically, a trocar assembly includes a cannula and an obturator. The cannula remains in place for use during the laparoscopic procedure, and the obturator includes a tip for penetrating body tissue. In endoscopic surgical procedures, surgery is performed in any hollow organ or tissue of the body through a small incision or through a narrow endoscopic tube (e.g., a cannula) inserted through a small entrance wound in the skin. In laparoscopic procedures, surgical operations in the abdomen are performed through small incisions (usually about 0.5 to about 1.5 cm). Laparoscopic and endoscopic procedures often require the surgeon to act on organs, tissues and vessels far removed from the incision. Depending on the type of procedure and the size of the patient, for instance, the surgeon selects a particular length of cannula they believe will be required for the surgery.

Accordingly, it may be helpful to provide an access device, or cannula, having an adjustable length to make the cannula adaptable to a broad range of patients and procedures.

### SUMMARY

The present disclosure relates to a surgical access device including a cannula body and a sleeve. The cannula body includes a housing, an elongated portion extending distally from the housing, and at least one pin extending radially outward from the elongated portion. The elongated portion defines a longitudinal axis and defines a channel extending therethrough. The sleeve is disposed in mechanical cooperation with the elongated portion of the cannula body. The sleeve defines a track for slidingly engaging the at least one pin of the cannula body, and is slidable along the longitudinal axis relative to the cannula body between a first position corresponding to a first length of the surgical access device and a second position corresponding to a second length of the surgical access device.

In aspects, the track of the sleeve may include a longitudinal section and a plurality of slots extending from the longitudinal section. The at least one slot of the plurality of slots may extend perpendicularly from the longitudinal section of the track. In aspects, each slot of the plurality of slots may extend perpendicularly from the longitudinal section of the track. Additionally, at least one slot of the plurality of slots may include a first portion and a second portion, where the first portion is closer to the longitudinal section than the second portion and defines a narrower width than the second portion. Each slot of the plurality of slots may include a first portion and a second portion, where the first portion is closer to the longitudinal section than the second portion and defines a narrower width than the second portion.

In aspects, at least two pairs of adjacent slots of the plurality of slots may be spaced equally apart. It is further disclosed that at least two slots of the plurality of slots may be spaced 0.5 inches apart.

In aspects, each pair of adjacent slots of the plurality of slots may be spaced equally apart, such as 0.5 inches apart.

In aspects, the surgical access device may also include a seal, such as an O-ring, disposed between the cannula body and the sleeve.

In additional aspects, the sleeve may be slidable along the longitudinal axis relative to the cannula body between a plurality of discrete positions, such as between at least three discrete positions.

The present disclosure also relates to a method of adjusting a length of a surgical access device. The method includes rotating a sleeve of the surgical access device in a first direction relative to a cannula body of the surgical access device, longitudinally translating the sleeve relative to the cannula body, and rotating the sleeve in a second direction relative to the cannula body.

In aspects, rotating the sleeve in the first direction may move a pin of the cannula body from a first slot of a track of the sleeve to a longitudinal channel of the track of the sleeve. It is also disclosed that longitudinally translating the sleeve relative to the cannula body may move the pin of the cannula body within the longitudinal channel of the track of the sleeve. It is further disclosed that rotating the sleeve in the second direction relative to the cannula body may move the pin from the longitudinal channel of the track of the sleeve into a second slot of the track of the sleeve. Rotating the sleeve in the second direction relative to the cannula body may produce at least one of audible feedback or tactile feedback.

### DESCRIPTION OF THE DRAWINGS

Various embodiments of the present disclosure are illustrated herein with reference to the accompanying drawings, wherein:

FIG. 1 is a perspective view of an adjustable length cannula in a locked position and at a first length;

FIG. 2 is an assembly view of the adjustable length cannula of FIG. 1;

FIG. 3 is an end cross-sectional view of the adjustable length cannula taken along section line 3-3 in FIG. 1;

FIG. 4 is a side cross-sectional view of the adjustable length cannula taken along section line 4-4 in FIG. 3;

FIG. 5 is an enlarged view of the area of detail indicated in FIG. 1;

FIG. 6 is a perspective view of a portion of the adjustable length cannula of FIGS. 1-5 in an unlocked position and at the first length;

FIG. 7 is a perspective view of a portion of the adjustable length cannula of FIGS. 1-6 in an unlocked position and at a second length;

FIG. 8 is a perspective view of a portion of the adjustable length cannula of FIGS. 1-6 in a locked position and at the second length; and

FIG. 9 is a perspective view of the adjustable length cannula of FIGS. 1-6 in the locked position and at the second length.

### DETAILED DESCRIPTION

Aspects of the presently disclosed adjustable length cannula will now be described in detail with reference to the drawings wherein like numerals designate identical or corresponding elements in each of the several views. As is common in the art, the term “proximal” refers to that part or component closer to the user or operator, i.e. surgeon or physician, while the term “distal” refers to that part or component farther away from the user.

Generally, the adjustable length cannula, often part of a trocar assembly, may be employed during surgery (e.g., laparoscopic surgery) and may provide for the sealed access of laparoscopic surgical instruments into an insufflated body cavity, such as the abdominal cavity. As will be described in additional detail below, the adjustable length cannula of the present disclosure is usable with an obturator insertable therethrough. The adjustable length cannula and obturator are separate components but are capable of being selectively connected together. For example, the obturator may be inserted into and through the adjustable length cannula until the handle of the obturator engages, e.g., selectively locks into, a proximal housing of the adjustable length cannula. In this initial position, the trocar assembly is employed to tunnel through an anatomical structure, e.g., the abdominal wall, either by making a new passage through the structure or by passing through an existing opening through the structure. Once the trocar assembly has tunneled through the anatomical structure, the obturator is removed, leaving the adjustable length cannula in place in the structure, e.g., in the incision created by the trocar assembly. The proximal housing of the adjustable length cannula may include seals or valves that prevent the escape of insufflation gases from the body cavity, while also allowing surgical instruments to be inserted into the body cavity.

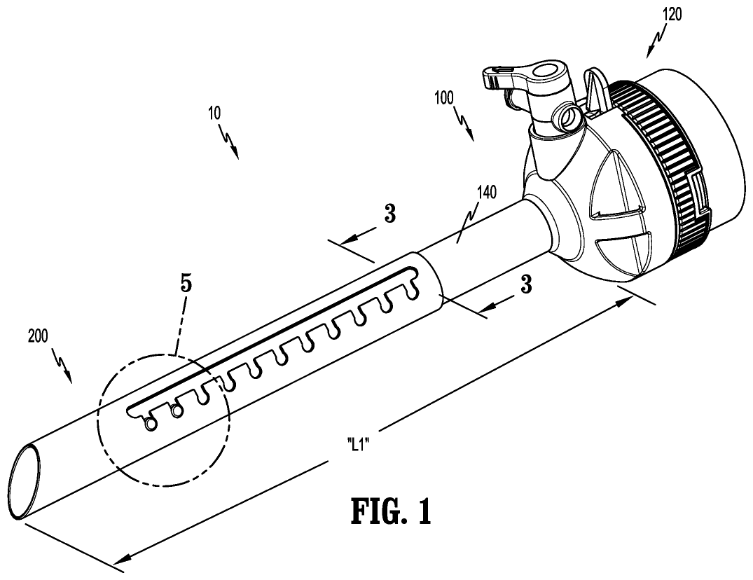

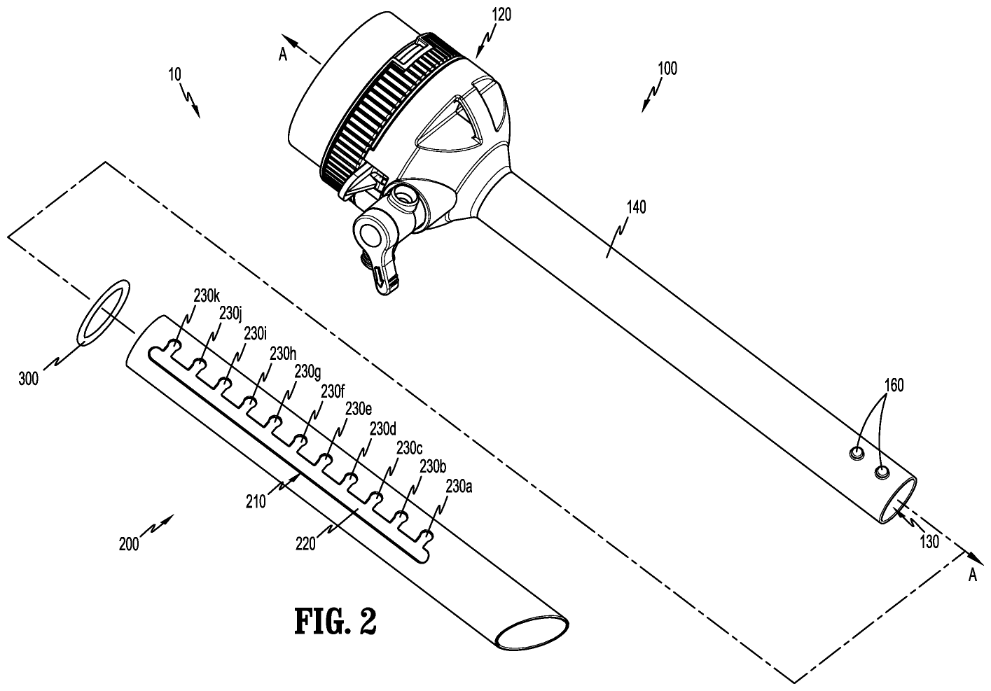

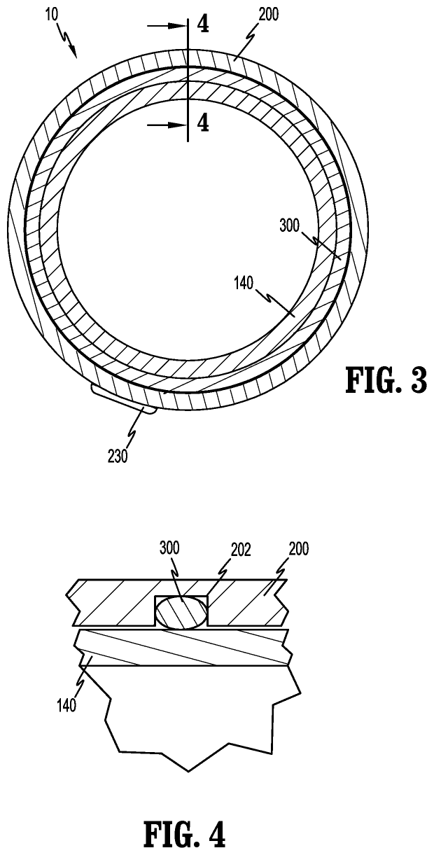

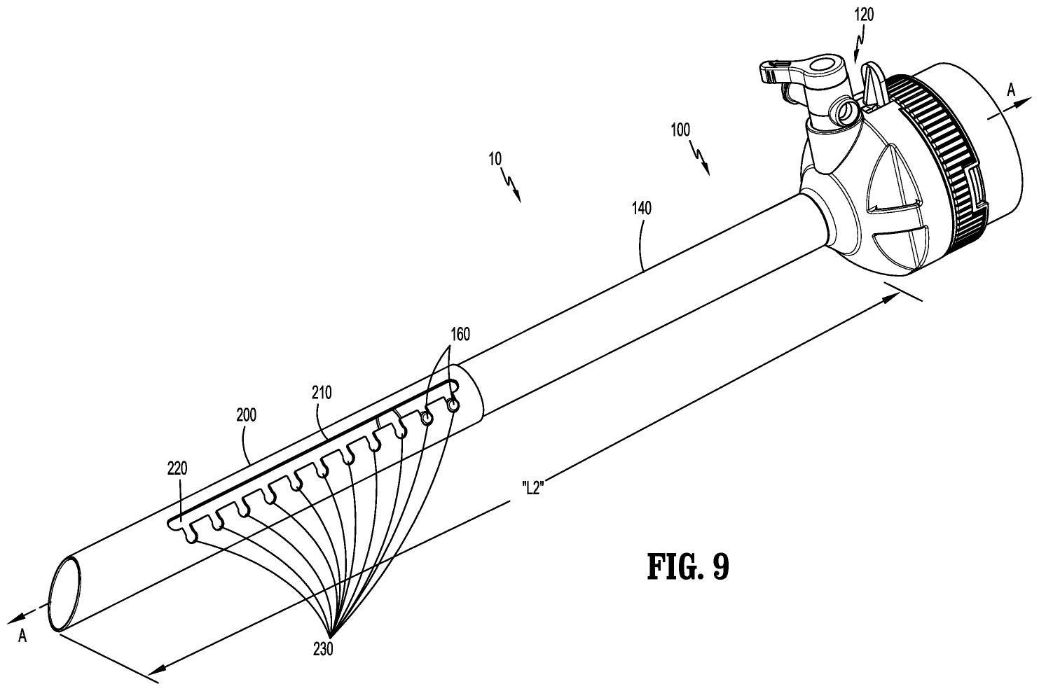

FIGS. 1-9 illustrate an exemplary surgical access device according to the present disclosure. With initial reference to FIG. 1, the surgical access device or adjustable length cannula 10 includes a cannula body 100 and a sleeve 200. The cannula body 100 includes a proximal housing 120 at its proximal end, and includes an elongated portion 140 extending distally from the proximal housing 120. The elongated portion 140 defines a channel 130 (FIG. 2) extending therethrough, and defines a longitudinal axis “A-A.” An obturator (not shown) is insertable through the channel 130 and is engagable with the housing 120, for instance. The sleeve 200 slidingly engages the elongated portion 140 of the cannula body 100. In particular, the sleeve 200 is incrementally slidable relative to the cannula body 100 between a first position (FIG. 1), where the elongated portion 140 and the sleeve 200 together form a first, shorter length “L1,” and a second position (FIG. 9), where the elongated portion 140 and the sleeve 200 together form a second, longer length “L2.”

Referring now to FIG. 2, the cannula body 100 includes at least one pin or projection 160 extending radially outward from the elongated portion 140. While two pins 160 are shown, the cannula body 100 may include more or fewer pins 160 without departing from the scope of the disclosure. The sleeve 200 includes a track 210 configured to slidingly engage the pins 160. More particularly, the track 210 of the sleeve 200 includes an elongated or longitudinal channel 220, and a plurality of slots 230a-230k extending at an angle (e.g., about) 90° from the longitudinal channel 220. The slots 230a-230k may also extend from the longitudinal channel 220 at angles greater than or less than 90°. While eleven slots 230a-230k are shown, the track 210 may include more or fewer slots 230 without departing from the scope of the disclosure.

With reference to FIGS. 2-4, a seal 300, such as an O-ring, is disposed between the elongated portion 140 of the cannula body 100 and the sleeve 200 to help ensure a sealed engagement therebetween. As particularly shown in FIG. 4, the sleeve 200 includes an annular recess or groove 202 for accepting the seal 300.

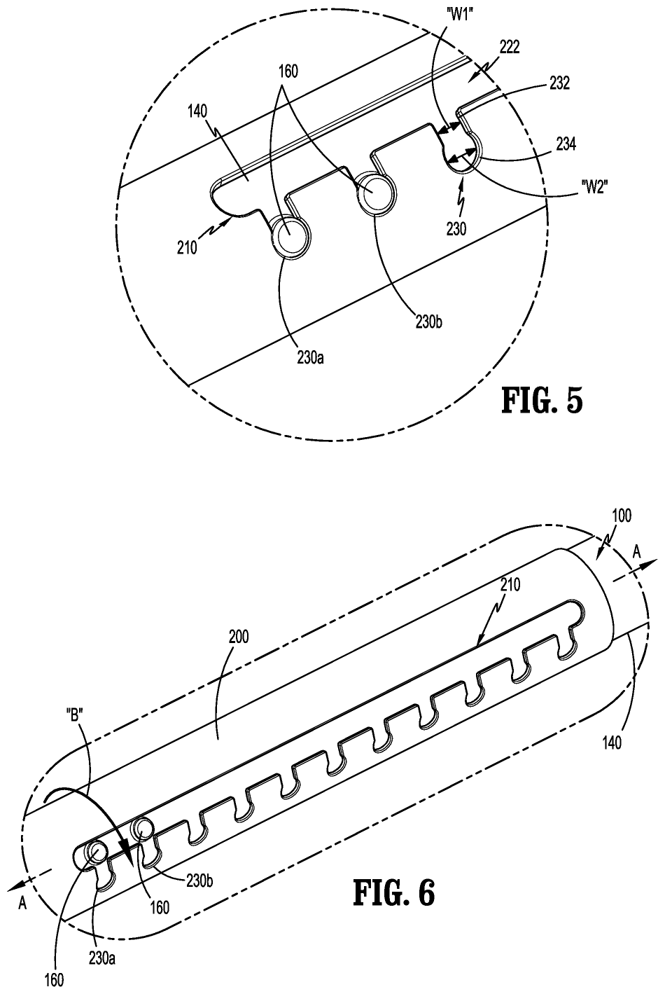

Referring now to FIGS. 5-8, further details of the adjustable length cannula 10 are shown. For instance, the engagement between the pins 160 and the track 210 are shown. With particular reference to FIG. 5, each slot 230a-230k is configured to releasably retain one pin 160. The inclusion of two pins 160 (as opposed to one pin 160) increases the robustness of the engagement between the cannula body 100 and the sleeve 200. When the pins 160 are retained within a respective slot (e.g., 230a and 230b), the length of the adjustable length cannula 10 is fixed (FIG.5).

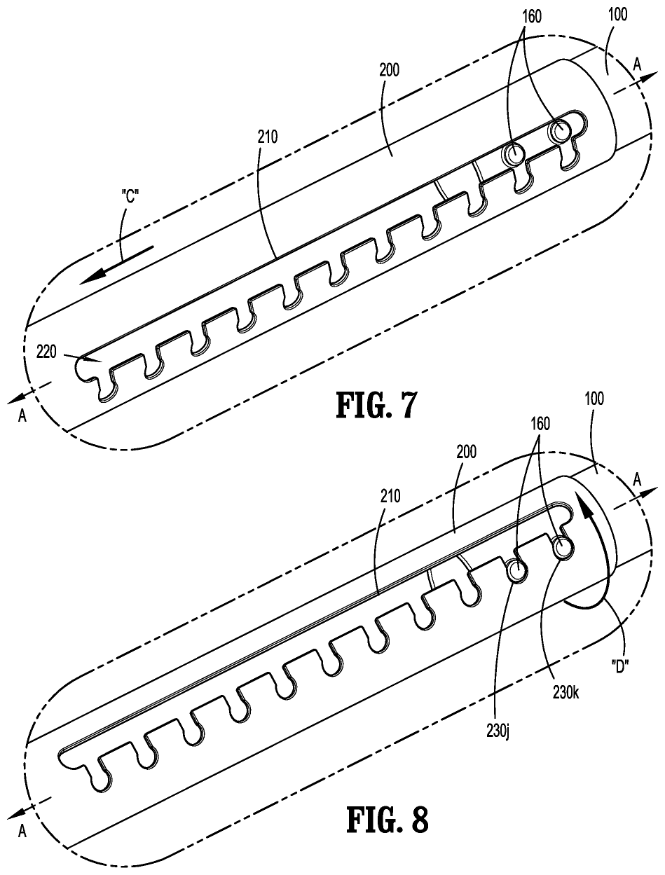

The method of adjusting the length of the adjustable length cannula 10 is shown in FIGS. 6-8. First, to move the pins 160 out of the slots 230, a user rotates or twists the sleeve 200 about the longitudinal axis “A-A” in the general direction of arrow “B” relative to the cannula body 100 (FIG. 6). Next, the user slides the sleeve 200 longitudinally in the general direction of arrow “C” (or the opposite direction) relative to the cannula body 100 (FIG. 7). This longitudinal sliding causes the pins 160 to travel within the longitudinal channel 220 of the track 210. Then, once the desired overall length of the adjustable length cannula 10 is achieved, the user rotates or twists the sleeve 200 about the longitudinal axis “A-A” in the general direction of arrow “D” relative to the cannula body 100 (FIG. 8). This rotation forces the pins 160 into desired slots 230 (e.g., slots 230j and 230k in FIG. 8), which fixes the longitudinal position of the sleeve 200 relative to the cannula body 100, thereby fixing the overall length of the adjustable length cannula 10.

The size and shape of the slots 230a-230k help direct and/or retain the pins 160 therein. That is, as shown in FIG. 5, each slot 230 includes a first portion 232, which is closest to the longitudinal channel 220 of the track 210, and a second portion 234. The first portion 232 defines a first width “W1,” which is narrower than a second width “W2” defined by the second portion 234. The smaller width of the first portion 232 of the slot 230 makes it more difficult for the pin 160 to enter into the first portion of the slot 232 from the longitudinal channel 220 of the track 210 and from the second portion 234 of the slot 230. Thus, the likelihood that the pin 160 inadvertently exits the slot 230 is reduced. Further, it is envisioned that the first width “W1” defined by the first portion 232 of the slot 230 is approximately the same as (e.g., +/−5%) a width of a portion of the pin 160 that is in contact therewith. In such devices, audible feedback and/or tactile feedback may occur (e.g., a click or snap) when the pin 160 enters the second portion 234 of the slot 230, for instance. Additionally, while a particular shape of the slots 230 is shown, the slots 230 may define different shapes, including regular or irregular shapes that are narrower adjacent the longitudinal channel 220 of the track 210, for instance.

The amount of space between adjacent slots 230 determines the increments between discrete positions in which the adjustable length cannula 10 can be adjusted. For instance, each of the adjacent slots (e.g., 230a and 230b) may be spaced 0.5 inches apart. Further, all the slots 230a-230k may be equally spaced from an adjacent slot (e.g., 0.5 inches between each adjacent slot), or the spacing between some adjacent slots may differ. In such a device, the spacing between adjacent slots (e.g., 230a-230d) may be smaller than the spacing between adjacent slots (e.g., 230e-230k) to allow for finer control of the length of the adjustable length cannula 10 when the adjustable length cannula 10 has a relative small length (FIG. 1). Here, the cannula body 100 may include one pin 160.

The present disclosure also relates to a method of adjusting the length of a cannula. The method includes rotating the sleeve 210 of the adjustable length cannula 10 in a first direction relative to the cannula body 100 to enable an adjustment of the length of the adjustable length cannula 10, longitudinally translating the sleeve 210 relative to the cannula body 100, and rotating the sleeve 210 in a second direction relative to the cannula body 100 to releasably lock the longitudinal position of the sleeve 210 relative to the cannula body 100.

While the above description contains many specifics, these specifics should not be construed as limitations on the scope of the present disclosure, but merely as illustrations of various embodiments thereof. Therefore, the above description should not be construed as limiting, but merely as exemplifications of various embodiments. Those skilled in the art will envision other modifications within the scope and spirit of the claims appended hereto.

What is claimed is:

1. A surgical access device, comprising:
a cannula body including a housing, an elongated portion extending distally from the housing, and at least one pin extending radially outward from the elongated portion, the elongated portion defining a longitudinal axis and defining a channel extending therethrough; and
a sleeve disposed in mechanical cooperation with the elongated portion of the cannula body, the sleeve defining a track for slidingly engaging the at least one pin of the cannula body, the sleeve being slidable along the longitudinal axis relative to the cannula body between a first position corresponding to a first length of the surgical access device and a second position corresponding to a second length of the surgical access device.

2. The surgical access device according to claim 1, wherein the track of the sleeve includes a longitudinal section and a plurality of slots extending from the longitudinal section.

3. The surgical access device according to claim 2, wherein at least one slot of the plurality of slots extends perpendicularly from the longitudinal section of the track.

4. The surgical access device according to claim 2, wherein each slot of the plurality of slots extends perpendicularly from the longitudinal section of the track.

5. The surgical access device according to claim 2, wherein at least one slot of the plurality of slots includes a first portion and a second portion, the first portion being closer to the longitudinal section than the second portion and defining a narrower width than the second portion.

6. The surgical access device according to claim 2, wherein each slot of the plurality of slots includes a first portion and a second portion, the first portion being closer to the longitudinal section than the second portion and defining a narrower width than the second portion.

7. The surgical access device according to claim 2, wherein at least two pairs of adjacent slots of the plurality of slots are spaced equally apart.

8. The surgical access device according to claim 2, wherein at least two slots of the plurality of slots are spaced 0.5 inches apart.

9. The surgical access device according to claim 2, wherein each pair of adjacent slots of the plurality of slots is spaced equally apart.

10. The surgical access device according to claim 9, wherein each pair of adjacent slots of the plurality of slots is spaced 0.5 inches apart.

11. The surgical access device according to claim 1, further including a seal disposed between the cannula body and the sleeve.

12. The surgical access device according to claim 11, wherein the seal is an O-ring.

13. The surgical access device according to claim 1, wherein the sleeve is slidable along the longitudinal axis relative to the cannula body between a plurality of discrete positions.

14. The surgical access device according to claim 1, wherein the sleeve is slidable along the longitudinal axis relative to the cannula body between at least three discrete positions.

15. A method of adjusting a length of a surgical access device, comprising:
rotating a sleeve of the surgical access device in a first direction relative to a cannula body of the surgical access device;
longitudinally translating the sleeve relative to the cannula body; and
rotating the sleeve in a second direction relative to the cannula body.

16. The method according to claim 15, wherein rotating the sleeve in the first direction moves a pin of the cannula body from a first slot of a track of the sleeve to a longitudinal channel of the track of the sleeve.

17. The method according to claim 16, wherein longitudinally translating the sleeve relative to the cannula body moves the pin of the cannula body within the longitudinal channel of the track of the sleeve.

18. The method according to claim 17, wherein rotating the sleeve in the second direction relative to the cannula body moves the pin from the longitudinal channel of the track of the sleeve into a second slot of the track of the sleeve.

19. The method according to claim 18, wherein rotating the sleeve in the second direction relative to the cannula body produces at least one of audible feedback or tactile feedback.
